(19) United States
(12) Patent Application Publication
(10) Pub. No.: US 2009/0209489 A1
(43) Pub. Date: Aug. 20, 2009

(54) Compositions for the treatment of diabetis, system PH disorders, high blood pressure, and a cancer marker system

(76) Inventor: Roger Wayne Brown, Phoenix, AZ (US)

Correspondence Address: Roger W. Brown, Ph.D., 810 W. Villa Maria Dr., Phoenix, AZ, 85023, US

(21) Appl. No.: 12/286,090

(22) Filed: Sep. 29, 2008

Related U.S. Application Data
(63) Continuation-in-part of application No. 12/070,313, filed on Feb. 19, 2008.

(51) Int. Cl. A61K 31/7008; A61K 31/7004; C12Q 1/34; A61P 35/00

(52) U.S. Cl. 514/62; 514/23; 435/18

(57) ABSTRACT

In accordance with the present invention, there is to provide a dietary supplement that provides a mammal with the essential carbohydrates needed to maintain proper health and functionality, to fend off illness, to restore proper system pH, to provide a new treatment for pH based illnesses, to provide a new marker system for the early detection of cancer, to provide a new treatment for borderline diabetics, to provide a new treatment for high blood pressure, to lessen the aging process of cells and to provide pets with another level of medications equal to that for humans.

### RELATED APPLICATIONS

This application claims priority on patent application Ser. No. 12/218,971 filed with the PTO on Jul. 21, 2008, patent application Ser. No. 12/151,394 filed with the PTO on May 7, 2008, patent application Ser. No. 12/079,907 filed with the US PTO on Mar. 31, 2008 and on patent application Ser. No. 12/070,313 filed Feb. 19, 2008, and is a continuation-in-part of patent application Ser. No. 12/070,313 filed Feb. 19, 2008, the entire disclosure of which is incorporated herein.

The present application is related to U.S. Pat. No. 6,339,076, issued Jan. 15, 2002, included by reference herein.

The present application is related to U.S. Pat. No. 7,183,269, issued Feb. 27, 2007, included by reference herein.

The present application is related to U.S. Pat. No. 5,681,857, issued Oct. 28, 1997, included by reference herein.

The present application is related to U.S. Pat. No. 4,043,757, issued Aug. 23, 1977, included by reference herein.

The present application is related to U.S. Pat. No. 7,202,220 B2, issued Apr. 10, 2007, included by reference herein.

The present application is related to U.S. Pat. No. 7,199,104 B2, issued Apr. 3, 2007, included by reference herein.

The present application is related to U.S. Pat. No. 7,196,064 B2, issued Mar. 27, 2007, included by reference herein.

The present application is related to U.S. Pat. No. 7,157,431 B2, issued Jan. 2, 2007, included by reference herein.

The present application is related to U.S. Pat. No. 6,929,807 B1, issued Aug. 16, 2005, included by reference herein.

The present application is related to U.S. Pat. No. 3,890,438 A, issued Jun. 1, 1975, included by reference herein.

The present application is related to U.S. Pat. No. 3,947,601 A, issued Mar. 1, 1976, included by reference herein.

The present application is related to U.S. Pat. No. 4,260,603 A, issued Apr. 1, 1981, included by reference herein.

The present application is related to U.S. Pat. No. 4,466,958 A, issued Aug. 1, 1984, included by reference herein.

The present application is related to U.S. Pat. No. 4,777,045 A, issued Oct. 1, 1988, included by reference herein.

The present application is related to U.S. Pat. No. 4,871,557 A, issued Oct. 1, 1989, included by reference herein.

The present application is related to U.S. Pat. No. 5,612,039 A, issued Mar. 1, 1997, included by reference herein.

The present application is related to U.S. Pat. No. 5,827,526, issued Oct. 1, 1998, included by reference herein.

The present application is related to U.S. Pat. No. 7,244,706, issued Jul. 17, 2007, included by reference herein.

The present application is related to U.S. Pat. No. 7,323,179, issued Jan. 29, 2008, included by reference herein.

The present application is related to U.S. Pat. No. 6,235,407, issued May 22, 2001, included by reference herein.

The present application is related to U.S. Pat. No. 6,972,180, issued Dec. 6, 2005, included by reference herein.

The present application is related to U.S. Pat. No. 6,670,141, issued Dec. 30, 2003, included by reference herein.

The present application is related to U.S. Pat. No. 6,294,349, issued Sep. 25, 2001, included by reference herein.

US Class: 514/8; 514/23; 514/54; 514/62; 424/725

### FIELD OF THE INVENTION

The present invention relates to the field of dietary supplements promoting good nutritional health and, more particularly, to the compositions of carbohydrates as dietary supplements that are required by mammals for good health and specifically to restore proper system pH, to provide a new treatment for pH based illnesses, to provide a new treatment for borderline diabetics, to provide a new treatment for high blood pressure and to provide a marker detection system for early detection of cancer.

### BACKGROUND OF THE INVENTION

Mammal's bodies produce a large number of different types of chemicals that the body uses to ward off disease, retard cell degradation, maintain memory and maintain overall body health. These chemicals are produced as a byproduct of what the mammal has eaten. If all of the right foods are eaten in the proper amounts then the body will produce enough of all of the chemicals required to keep it functioning properly. Over the years people have sought after which chemicals are actually necessary for good health and which ones are just good. As this field is evolving more and more information is being discovered about what chemicals mammal's bodies require for proper functionality.

Over the last ten years a lot of research has been done concerning cell communications and its importance to a properly functioning mammal's body. This research indicates that there are eight essential sugars that all mammals need in order to stay healthy; http://www.glyconutrients-center.org/ and http://www.glyconutrientsreference.com/. These Essential Sugars, also called carbohydrates, are know to researchers as glycoproteins and they coat cell membranes in all mammal bodies. Six of these carbohydrates (sugars) are generally missing in the diets of most humans and seven are missing from the diet of animals. However, very small concentrations of these missing carbohydrates are contained in various plants and some sea products. A synopsis of these seven essential sugars/carbohydrates and what functions they have been found to influence follows:

D-Galactose is readily available in human diets but not in animal diets. It is obtained from the conversion of lactose (milk sugar) and is also easily obtained from dairy products UNLESS you suffer from lactose intolerance or are a vegetarian who does not eat dairy products.

D-Mannose is not readily available in our diets. The most popular source is Aloe Vera. It is also available in tiny quantities in the bran of whole wheat. However, it is very unstable and must be taken fresh from the plant and properly standardized to be of any benefit. It plays a profound role in cellular interactions and has even been known to lower blood sugar levels. It is absolutely vital to proper immune defenses against microbial invaders and has a natural and powerful anti-inflammatory effect. This sugar is readily available in supplemental form. Good for: Wound healing, Diabetics, Anti-viral, Anti-inflammatory and Arthritis.

N-Acetyl-Glucosamine is not readily available in our diets. It is particularly beneficial for cartilage regeneration and joint inflammation. Glucosamine derivatives are well-known natural medicine for arthritic conditions comes from this sugar compound. It has many more therapeutic effects and deficiencies or malfunctions of this sugar have been linked to diseases of the bowel. Derivatives of this sugar are readily available in supplemental form. Good for: Wound repair, Range of motion, Insulin production, Arthritic conditions, Learning, HIV and Vision.

L-Fucose is not readily available in our diets but is readily found in breast milk, astragalus herb, in several medicinal mushrooms, and in certain brown algae. It has numerous well-documented benefits for the immune system and has been shown to inhibit some cancer growth and metastasis. Good for: Long term memory, Cancer and tumors, Group 1 anti-viral medication, and Skin allergies.

D-Xylose is not readily available in our diets. It is often seen in sugarless gums, candies, etc. in that it has a sweet taste but does not cause tooth decay. It has recently been added to nasal sprays and appears to discourage the binding of allergens and pathogens to mucous membranes. It also has known anti-bacterial and fungal properties and may help prevent certain cancers. Good for: Anti-fungal and gram negative bacteria.

N-Acetyl-Neuraminic Acid is not readily available in our diets but is another sugar that abounds in breast milk and dramatically impacts brain function and growth. It, too, boosts immune function and has documented anti-viral actions. Interestingly, in certain disease states, the ability to digest this sugar is impaired. Good for: Best Group 5 Anti-viral known, Kidney stones, Asthma, Learning and Arthritis.

N-Acetyl-Galactosamine is not readily available in our diets. It is the least known of the essential sugars although it appears to inhibit the growth of some tumors and, like the other sugars, plays an individual role in keeping cellular messages clear and promptly delivered. Most of these sugars do not involve or require insulin for their use and go directly to the cells where they are incorporated into the cell structure wherever they are needed. Good for: Heart disease, Aging (cell rejuvenation), Joint functioning, blood pressure, glucose, system pH, and Vision.

The actual body requirements for these missing carbohydrates has been hard to estimate because of their rarity and because of this the FDA has not set a lower daily intake limit on any of them. However, research has indicated a level of dose for each of these carbohydrates required to produce noticeable effects. The base level for each of these carbohydrates is about 0.005 mg/kg of body weight or 0.4 mg/day for a 150 pound mammal. These levels correspond to base levels of other medications.

The base level is not the required level to start seeing results but the level below which nothing much has been seen. The general therapeutic levels are above 0.1 mg/kg of body weight or 8 mg for a 150 pound mammal. These findings indicate that the minimum required level for these essential carbohydrates is a hundred times larger than is available in natural foods including the specially prepared supplements designed to alleviate the missing carbohydrate deficiency.

The seven essential sugars/carbohydrates/glycoproteins are: D-Galactose, L-Fucose, D-Mannose, D-Xylose, N-Acetyl-Glucosamine, N-Acetyl-Neuraminic Acid, and N-Acetyl-Galactosamine. Of these seven carbohydrates D-Mannose and Glucosamine derivatives are available as full strength supplements from a large number of over the counter drug stores and D-Galactose is available from specialty suppliers. The remaining four are much too expensive for companies to currently package in full strength so all that is generally available to the public are very low concentration food substitutes.

While there are several different companies selling glyconutrients only one of them has filed for patent protection in the US. Glyconutrient is a coined marketing term which defines these glycoproteins when in their less than pure herbal form. This company is Mannatech, Inc. of Coppell Tex. and they sell their glyconutrients through a chain of 500,000 independent dealers worldwide. They filed an initial U.S. patent application in 1997 and was granted a U.S. patent on Aug. 16, 2005; U.S. Pat. No. 6,929,807 B1. Since then they have filed four additional amendments to this one patent. They were granted additional patent numbers which are: U.S. Pat. No. 7,157,431 B2, U.S. Pat. No. 7,196,064 B2, U.S. Pat. No. 7,199,104 B2, and U.S. Pat. No. 7,202,220 B2.

Their first patent sets out eight essential sugars and ties these sugars to food sources where they can be found. However, they don't disclose the actual amount of the various essential sugars/carbohydrates in these food sources. Their web site is located at: https://www.mannatech.com/Default.aspx

They produce specific supplements used to address several different conditions that arise in mammals: weight management, alcoholism, nutrition, wellness management, lifestyle management, growth essentials, performance management, skin care and performance nutrition. All of these products are different mixes of the same basic foods and they contain very little of what is required by the body to function properly.

Another company also selling glyconutrients is shown here: http://www.naturalcureguide.com/glyconutrients.html Again, as Mannatech does, this company also provides the greatest amounts of the carbohydrates that are not in short supply in the body.

While all of the companies selling glyconutrients have products specifically orientated to correct certain illnesses none of them have anything that addresses the common cold, cold remedies, memory or cell aging. Clearly these missing carbohydrates have capabilities in these areas but these areas are not being addressed by any of the current glyconutrient companies.

One of the short comings with the current offerings to the public is the lack of these missing carbohydrates at therapeutic dose levels (levels at which changes are seen in hours instead of many months). For example, L-Fucose is available in Gum Tragacanth ($34/pound) and Brewer's Yeast (half a pound for $6), but the availability of L-Fucose in Gum Tragacanth is only 0.1% by weight and only 0.05% in Brewer's Yeast. Currently available glyconutrient supplements supply less than 0.1 mg of L-Fucose per daily dose for a 150 pound mammal.

N-Acetyl-Galactosamine is available in shark cartilage (3 oz. $16) but there is only 0.01% by weight of it there. By taking the specially made glyconutrient supplements the daily dose of N-Acetyl-Galactosamine is still generally under 0.1 mg. Likewise, N-Acetyl-Neuraminic Acid is available in Whey Protein (36 26 gram servings for $33) and Hen's eggs but there is only 0.02% of it there by weight. A daily dose from one of the special glyconutrient supplements generally has less than 0.2 mg of it available.

As these glyconutrients are mainly herbal supplements a large part of the population are allergic to their ingredients. Aloe Vera and Shark Cartilage are two necessary herbal type ingredients that are necessary in these mixes but they can cause severe allergic reactions when ingested by many people.

Mannatech even admits that their formulations are extremely weak and require many months to see any results at all “Be patient! Research shows that it may take up to 4 months (or more) to notice the effects of any changes you make to your diet.” https://www.mannatech.com/Shopping/Product.aspx and also see https://www.mannatech.com/Shopping/RDReports.aspx

There have been numerous complaints that Manntech products do not provide any benefit at all even after months of usage; http://www.reviewcentre.com/reviews94020.html “I saw Mannatech Ambrotose, Glyconutrients advertised on a Fibromyalgia site. I was contacted by a Mannatech Associate and was advised that Ambrotose could help with all my ailments—Fibromyalgia, Osteoarthritis, Asthma, allergies etc. I was also told that I wouldn't need to continue to take the vitamin, mineral and herbal supplements I had been using. Now, 4 months on, my asthma has worsened, my cholesterol levels have gone up and my fibromyalgia and arthritis are unchanged.”

From this it can be seen that most of the currently available special glyconutrient supplements sold to replace these missing carbohydrates contain mainly filler material and other chemicals that are already available to the body through other sources or are not needed. Additionally, many of these special supplements contain ingredients known to excite an allergic reaction in a large part of the population; such as Whey Powder and Aloe Vera ingredients. On the other hand there is no known allergic reaction to these essential sugars when taken in their pure form and at therapeutic dose levels.

One of the essential sugars (a carbohydrate), N-Acetyl-Neuraminic Acid (Sialic Acid), has been shown to be more than 1000 times more effective at killing Group 5 viruses than any other known medication when used in therapeutic doses “Another study reported in a 1995 issue of Antimicrobial Agents and Chemotherapy, stated that a sialic acid mixture was up to 1000 times more effective in fighting influenza than potent antiviral drugs. These viruses can cause the common cold as well as the flu.” http://www.glyconutrients-center.org/N-acetylneuraminic-acid.php

Even though the effectiveness of Sialic Acid as an antiviral agent been known for over a decade it is still waiting to be offered to the public in therapeutic dose levels.

When all of the body systems are functionally correctly and have the proper level of nutrients the body is healthy. When the body's supply of required chemicals and nutrients is low, or deficient, the body becomes sick. As the levels of these missing chemicals and nutrients degrades further the body becomes very sick.

One such deficiency disease is high Blood Pressure (BP). The risk of cardiovascular disease increases progressively throughout the range of arterial pressure, beginning at 115/75 mm Hg. In the past, hypertension was only diagnosed if secondary signs of high arterial pressure were present, along with a prolonged high systolic pressure reading over several visits. In the US, this reactive stance has been soundly rejected in light of recent evidence. However in the UK, patients' readings are still considered normal up to 140/90 mm Hg.

Clinical trials demonstrate that people who maintain arterial pressures at the low end of these pressure ranges have much better long term cardiovascular health. The principal medical debate concerns the aggressiveness and relative value of methods used to lower pressures into this range for those who do not maintain such pressure on their own. Elevations, more commonly seen in older people, though often considered normal, are associated with increased morbidity and mortality. The clear trend from double blind clinical trials (for the better strategies and agents) demonstrates that lower arterial pressure correlates with lower rates of disease.

In individuals older than 50 years, hypertension is considered to be present when a person's systolic blood pressure is consistently 140 mm Hg or greater. Beginning at a systolic pressure of 115 and diastolic pressure of 75 (commonly written as 115/75 mm Hg), cardiovascular disease (CVD) risk doubles for each increment of 20/10 mmHg. Prehypertension is defined as blood pressure from 120/80 mm Hg to 139/89 mm Hg. Prehypertension is not a disease category, rather, it is a designation chosen to identify individuals at high risk of developing hypertension. The Mayo Clinic specifies blood pressure is “normal if it's below 120/80”. Patients with blood pressures over 130/80 mm Hg along with Type 1 or Type 2 diabetes, or kidney disease require further treatment.

Resistant hypertension is defined as the failure to reduce BP to the appropriate level after taking a three-drug regimen. The American Heart Association released guidelines for treating resistant hypertension.

There are many classes of medications for treating hypertension, together called antihypertensives, which act by lowering blood pressure. Evidence suggests that reduction of the systolic blood pressure by 5-6 mm Hg can decrease the risk of stroke by 40%, of coronary heart disease by 15-20%, and reduces the likelihood of dementia, heart failure, and mortality from vascular disease.

The aim of treatment should be blood pressure control to <140/90 mm Hg for most patients, and lower in certain contexts such as diabetes or kidney disease (some medical professionals recommend keeping levels below 120/80 mm Hg). Each added drug may reduce the systolic, the larger of the two BP numbers, blood pressure by 5- 10 mm Hg so often multiple drugs are necessary to achieve blood pressure control; http://en.wikipedia.org/wiki/Hypertension

Commonly used drugs include:
ACE inhibitors such as creatine captopril, enalapril, fosinopril (Monopril), lisinopril (Zestril), quinapril, ramipril (Altace).
Angiotensin II receptor antagonists: eg, telmisartan (Micardis, Pritor), irbesartan (Avapro), losartan (Cozaar), valsartan (Diovan), candesartan (Amias).
Alpha blockers such as prazosin, or terazosin.
Beta blockers such as atenolol, labetalol, metoprolol (Lopressor, Toprol-XL), propranolol.
Calcium channel blockers such as nifedipine (Adalat) amlodipine (Norvasc), diltiazem, verapamil.
Direct renin inhibitors such as aliskiren (Tekturna).
Diuretics: eg, bendroflumethiazide, chlortalidone, hydrochlorothiazide (also called HCTZ), and
Combination products (which usually contain HCTZ and one other drug).
Doxazosin has been shown to increase risk of heart failure so it isn't being recommended for further use.

These medications all have associated adverse side effects and they can't be tolerated by all patients. The selection of medication for specific patients to reduce the BP more than 5-10 mm Hg can be a very risky process for the patient. Most of these medications only reduce the BP by 5 mm Hg while only a very few will reduce the BP up to 10 mm Hg. See page four of the print out from http://www.rxlist.com/cgi/generic/teraz_cp-page2.htm

While elevated blood pressure alone is not an illness, it often requires treatment due to its short term and long term effects on many organs. The risk is increased for:
Cerebrovascular accident (CVAs or strokes)
Myocardial infarction (heart attack)
Hypertensive cardiomyopathy (heart failure due to chronically high BP)
Hypertensive retinopathy—damage to the retina
Hypertensive nephropathy—chronic renal failure due to chronically high BP
Hypertensive encephalopathy—confusion, headache, convulsion due to vasogenic edema in brain due to high BP

The level of blood pressure regarded as deleterious has been revised down during years of epidemiological studies. A widely quoted and important series of such studies is the Framingham Heart Study carried out in an American town, Framingham, Mass. The results from Framingham and of similar work in Busselton, Western Australia, have been widely applied. To the extent that people are similar this seems reasonable, but there are known to be genetic variations in the most effective drugs for particular sub-populations.

Another deficiency disease is borderline diabetics. Here, the body has a hard time regulating the amount of glucose in the blood stream and glucose levels stay higher than normal but below true diabetic levels. While a high level of glucose in the blood is not dangerous in itself this condition generally gets worse with time and usually turns into diabetics. There is presently no medications for this condition.

Another deficiency disease is cancer. A result of a body being deficient of the required nutrients for an extended period of time is the formation of tumor cells. Unless these deficiencies are addressed this condition deteriorates into cancer. If left untreated the next stage is death of the organism.

A recent book, “The Calcium Factor” by Robert Barefoot, addresses research into this area. In his research he found out that as long as the body's pH was kept near 7 the body functioned normally and healthily. As the body pH became more acidic the body's condition slowly deteriorated. At pH levels of 5 and under cancer was progressive throughout the body.

As a treatment to low pH conditions he found that Calcium would raise the body pH and if done soon enough could reverse the disease. The reason for this was from the fact the cancer cells thrive in an acidic environment but they can't live in an alkaline environment (like a pH of 7). While normal cells thrive in an alkaline environment and do poorly in an acidic environment. By adding Calcium to the diet it was possible to raise the pH level of the body back to normal and the disease would go into remission.

They also found that as the body's pH became very low and cancer was found that simply adding Calcium to the diet did not raise the body's pH level as had been previously seen. Then they began to add vitamin D to the Calcium mix and again they had some success. However, at very low pH levels, <5, this didn't work either.

The problem was that as the body's pH became lower its ability to absorb Calcium decreased. The effect was logarithmic which meant for a small decrease in body pH it required a large intake of Calcium and vitamin D to raise the body pH back to the healthy level. For very low body pH levels Calcium therapy became dangerous itself because of the large doses required. Consequently, using Calcium therapy to cure cancer has not progressed any farther. What is needed is another way to raise the body's pH level without the need to add large amounts of Calcium to the diet.

A related matter complicated the detection of these cancers. It was how to measure the body pH consistently. The pH in the vicinity of the cell is the determining factor if cancer cells can grow or not, see Barefoot page 96. Additionally, the blood has its own pH level as does the lymphatic system and spinal fluid and saliva. While research found that using saliva pH as an indicator of Calcium deficiency worked well they did not go any further but instead turned all attention to Calcium therapy instead of refining an early warning system for cancer.

It is therefore an object of the invention to aid the body's ability to fight colds and infections by providing it with the correct mix and level of carbohydrates needed by the body to fend off the infection and to keep the body healthy.

It is another object of the invention to aid the body's ability to lessen the cells aging process by providing it with the correct mix and level of carbohydrates needed by the cells to keep them healthy.

It is another object of the invention to give an animal the ability to fight viruses and infections much more effectively by providing it with the correct mix and level of carbohydrates needed by its body.

It is another object of the invention to promote good health and wellness to all types of mammals through the proper mix of carbohydrates targeted to specific aliments, like increasing mental ability and learning, wound repair and long term memory.

It is another object of the invention to provide a new and safe treatment to reduce high blood pressure in mammals.

It is another object of the invention to provide a new and safe treatment for borderline diabetics.

It is another object of the invention to refine an early warning detection system for the early detection of cancer and cancerous conditions in the body.

It is another object of this invention to provide a new and safe treatment for cancer and cancerous conditions in the body.

It is another object of this invention to determine a new way to raise the body's pH level without the need to take large doses of Calcium in order to keep the body disease free.

### SUMMARY OF THE INVENTION

In accordance with the present invention, there is to provide a dietary supplement that provides a mammal with the essential carbohydrates needed to maintain proper health and functionality, to fend off illness, to restore proper system pH, to provide a new treatment for pH based illnesses, to provide a new treatment for high blood pressure, to provide a new marker test for early detection of cancer, to provide a new treatment for borderline diabetics, to lessen the aging process of cells and to provide pets with another level of medications equal to that for humans.

### BRIEF DESCRIPTION OF THE DRAWINGS

There are no drawings.

### DESCRIPTION OF THE PREFERRED EMBODIMENT

The carbohydrates included in the dietary supplement of the invention are available from a number of manufactures. Most are derived synthetically from other pure chemicals rather than being plant or animal derivatives. A supplier for the two most expensive essential sugars, Sialic Acid (CAS#131-48-6) and N-Acetyl-Galactosamine (CAS#1811-31-0), is R&S PharmChem located in China http://www.rspharmchem.com. A supplier for L-Fucose (CAS#2438-804) is AppliChem located in Germany http://www.applichem.de/perl/catalog/catalog.pl. AppliChem can also supply two other more readily available essential sugars, D-Galactose (CAS#59-23-4) and D-Xylose (CAS#58-86-6). D-Mannose (CAS#3458-28-4) is available from a number of the larger supplement suppliers like NOW Foods http://www.nowfoods.com/. The remaining carbohydrate, N-Acetyl-Glucosamine (CAS#98632-70-3) is generally used in one of its many derivative forms. This invention uses the derivatives Glucosamine HCL (CAS#66573-21-5) and Glucosamine Sulfate (CAS#29031-19-4) instead of Glucosamine (CAS#3416-24-8). Both of these carbohydrates are readily available at most drug stores.

It should be recognized that the composition of the carbohydrate is not intended to be limited by the source from which it is obtained.

It should be stressed that this invention does not incorporate the use of Glucose or Acetylated Mannose. While Glucose is one of the eight essential sugars it is so prevalent in today's diets that adding additional amounts of Glucose in a supplement generally provides no useful benefit. Acetylated Mannose is a plant derivative from the Aloe Vera plant that has not been shown by independent research to be of any beneficial use as a dietary supplement.

Although the present invention includes the above cited seven essential sugars (carbohydrates), it should be noted that other carbohydrates, nutritional compounds or biologically active or inert compounds can be included in the dietary supplement of the invention. Such other ingredients may include spices, flavorings, buffers, gels, binders, filler material, lubrication material, vitamins and or minerals and/or other such compounds that facilitate the formulation or administration of the inventive dietary supplement. These components can be provided separately to a mammal given said dietary supplement.

Many different types of vitamins and minerals can be included in the dietary supplement of the invention. While a few vitamins and minerals of synthetic origin do possess nutritional value, particular embodiments of the dietary supplement herein can contain nutritionally effective amounts of non-toxic vitamins and minerals obtained predominantly from natural sources.

Other compounds, agents and nutrients can also be included in the dietary supplement of the invention, for example: cellulose, calcium carbonate, stearic acid, amino acids, glycine, glycerine, essential fibers, essential oils, essential botanicals, essential enteric ecology and flora growth promoters, essential fatty acids, honey and enzymes.

Independent research indicates that these seven essential sugars are not stored in the body very long. After ingestion the sugars are assimilated into the blood stream within minutes (if taken on an empty stomach). Once in the blood stream they flow through the body and cells in need of these nutrients take what they need and the rest flows on. Most of these unused sugars are excreted via the urine within 12 hours after ingestion. The tests indicate that while excess of these sugars are excreted from the body within 12 hours the cells maintain a internal supply of these sugars for a period of up to a week. Studies have also shown that taking these seven essential sugars, in the levels covered by this invention, did not cause an abnormal rise in the blood sugar levels of diabetics.

The dietary supplement form of the invention has been prepared and can be administered to mammals in powdered, reconstitutable powder, liquid-solid suspension, liquid, capsule and tablet dosage forms. It should be readily obvious to one of ordinary skill in the science of formulations that the present dietary supplement can also be formulated appropriately for irrigation, ophthalmic, rectal, sublingual, transdermal buccal, vaginal, or dermal administration. Thus, other dosage forms such as chewable candy bar, concentrate, drops, elixir, emulsion, film, gel, granule, chewing gum, jelly, oil, paste, pastille, pellet, shampoo, rinse, soap, sponge, suppository, swab, syrup, chewable gelatin form, or chewable tablet can be used.

Due to varying diets among people, the dietary supplement of the invention can be administered in a wide range of dosages and formulated in a wide range of dosage unit strengths. For example, for those people who are missing seven of the eight essential carbohydrates from their diet a dietary supplement containing those carbohydrates in nutritionally effective amounts can be formulated. As well, for those people whose bioabsorption of essential carbohydrates is extremely efficient, a dietary supplement formulation containing reduced amounts of essential carbohydrates can be prepared.

It should be noted that the dosage of the dietary supplement can also vary according to a particular ailment or disorder that a mammal is suffering from when taking the supplement. For example, a person suffering from chronic colds will generally require a dose different than an animal would who is sick in order to obtain a benefit. An appropriate dose of the dietary supplement can be readily determined by monitoring patient response, i.e., general health, to particular doses of the supplement. As well, when another agent such as a vitamin and/or a herbal extract is being administered to a mammal along with the present carbohydrate dietary supplement, the appropriate doses of the supplement and each of the agents can be readily determined in a like fashion by monitoring patient response, i.e. general health, to particular doses of each.

It is contemplated by the invention that the dietary supplement can be administered simultaneously or sequentially in one or a combination of dosage forms. While it is possible and even likely that the present dietary supplement will provide an immediate overall health benefit, such benefit may take hours or days to materialize. Nonetheless, the present carbohydrate dietary supplement will provide a beneficial nutritional response in a mammal consuming it.

For the examples herein, the dietary supplement of the invention was administered as a powder-containing capsule. According to the capsule size and ingredients used in a given study exemplified herein, the dietary supplement was administered by oral ingestion. The indicated doses for humans in Example 1 are based upon #00 sized capsules.

### EXAMPLE 1

A suitable composition for a product according to the present invention is shown in the following table:

| | Weight % | | |
|---|---|---|---|
| Carbohydrate | (range) | Weight % (tested) | Human (mg) |
| D-Galactose | 0.1 to 40 | 6.3 | 43 |
| L-Fucose | 0.1 to 90 | 3.1 | 21 |
| D-Mannose | 10 to 70 | 52.1 | 358 |
| D-Xylose | 0.1 to 70 | 6.3 | 43 |
| Glucosamine HCL | 10 to 60 | 25.1 | 173 |
| Sialic Acid | 0.1 to 50 | 6.5 | 45 |
| N-Acetyl-Galactosamine | 0.1 to 90 | 0.6 | 4 |

In this combination the ingredients Glucosamine HCL, D-Galactose and D-Mannose are optional and preferred. Instead of using Rice Flour as an inactive filler as is done in most products currently available to the public the three optional ingredients set out above were used as active fillers. A disadvantage of using Rice Flour as an inactive filler is its high glycemic index tends to drive a mammal's Triglyceride levels up very high, while using only essential sugars as fillers doesn't.

The ingredients are typically in a powered form and are dry blended in a mixer. The mixture can then be packaged as a blended powder into capsules or caplets. In this example the mixture was packaged into size 00 capsules with an average weight of 687 mg for human doses and 25 mg for animal doses. The mg per ingredient for animals would be found by dividing the human ingredient dose in mg by 27.48, for example: for D-Mannose the animal ingredient dose would be 13.0 mg.

This composition is considered a health maintenance mix. It was intended to reduce the probability of infections, like colds, while taking the composition. There were three test trials run using this composition on both humans and animals. These three tests ran from late January 2007 to late December 2007. In the first test, that lasted for two weeks, a capsule/dose was administered twice a day for two weeks. The next test lasted for one month during which time one capsule/dose was administered per day at bedtime.

The third test lasted slightly over nine months and the dose was one capsule/dose a week administered at bedtime. During that time there were no deaths or adverse reactions to the composition by any test subjects either human or animal. Regular blood, lipid and electrolyte testing was done. A base line was run prior to the test and during the test blood testing was done regularly to determine if there were any adverse effects due to taking the composition.

The end result was that no one taking this composition contracted a cold or any other type of viral infection during the entire period of the test, even though one of the individuals in the test was prone to chronic colds and flu. This test extended through two different flu seasons and the test subjects continued to work everyday and come in contact with infected people on a daily basis yet they didn't catch anything while taking this composition.

In a follow up test these capsules were taken on a once a month basis by the same human test group during the flu season. Within two weeks following a monthly dose (this was during the four week pause after the end of the weekly tests) two of the subjects came down with a cold or flu. After this the test was stopped and no additional capsules were taken and within two weeks all the test subjects resumed their normal activity of catching colds as normal.

During the animal tests old cats were exposed to new cats infected with FPV on two occasions. Nothing special was done for the first exposure. The second time an additional dose was given just after exposure. After these two exposures, which were several months apart, all of the old cats tested positive for FPV. However, none of the cats came down with any symptoms and are in excellent health at the time of this writing.

### EXAMPLE 2

In a different embodiment of this invention another suitable composition for a product according to the present invention is shown in the following table:

| | Weight % | | |
|---|---|---|---|
| Carbohydrate | (range) | Weight % (tested) | Human (mg) |
| D-Galactose | 0.1 to 50 | 7.2 | 25 |
| L-Fucose | 0.1 to 90 | 14.4 | 50 |
| D-Mannose | 1.0 to 70 | 31.6 | 110 |
| D-Xylose | 0.1 to 70 | 10.8 | 38 |
| Glucosamine HCL | 1.0 to 50 | 10.8 | 38 |
| Sialic Acid | 0.1 to 50 | 10.8 | 38 |
| N-Acetyl-Galactosamine | 0.1 to 90 | 14.4 | 50 |

In this combination the ingredients Glucosamine HCL and D-Mannose are optional and preferred. In this composition D-Galactose is not considered to be optional as this composition is intended for animals as well as humans.

This composition is considered a wellness mixture. It was intended to reduce the probability of infections, like colds, while taking the capsules. These ingredients provide Anti-Viral, Anti-Fungal and defense against Gram Negative Bacteria. This combination was initially derived from the findings of independent researchers. In recent studies in 2008 this composition was also found to be extremely effective at mitigating the effects of handover due to over consumption of alcohol.

### EXAMPLE 3

In a different embodiment of this invention another suitable composition for a product according to the present invention is shown in the following table:

| Carbohydrate | Weight % (range) | Weight % (tested) | Human (mg) |
|---|---|---|---|
| D-Mannose | 1.0 to 40 | 28.8 | 100 |
| Glucosamine Sulfate | 0.1 to 30 | 20.8 | 72 |
| D-Xylose | 0.1 to 70 | 28.8 | 100 |
| Sialic Acid | 0.1 to 50 | 21.6 | 75 |

In this combination the ingredient D-Mannose is optional and preferred. This composition was packaged in a size #1 capsule and taken twice a day. This combination is good for both humans and animals.

This composition is considered a cold pill mix. It is intended to reduce the effects of viral infections, like colds, and speed recovery. These ingredients provide Anti-Viral, Anti-Fungal and defense against Gram Negative Bacteria the same as in the wellness mixture, shown in Example 2, except here the amounts have been raised to achieve the maximum beneficial effect.

When taken after the onset of a cold dramatic relief is felt within four hours of taking this composition. The two doses should be taken 12 hours apart and not within two hours of a meal. Generally 10 PM and 10 AM seemed to work best for the test subjects. In some cases just taking a single dose at bedtime was sufficient to completely end all of the symptoms by morning.

### EXAMPLE 4

In a different embodiment of this invention another suitable composition for a product according to the present invention is shown in the following table:

| | Weight % | | |
|---|---|---|---|
| Carbohydrate | (range) | Weight % (tested) | Human (mg) |
| L-Fucose | 0.1 to 80 | 22.1 | 75 |
| Glucosamine Sulfate | 0.1 to 50 | 36.8 | 125 |
| N-Acetyl-Galactosamine | 1.0 to 90 | 41.1 | 140 |

In this combination the ingredient Glucosamine Sulfate is optional. This composition was packaged in a size #1 capsule and taken once a week. It is good for both humans and animals. This composition is considered a blood pressure reducer mixture. This combination was derived from the findings of recent in-house human testing.

This composition has been shown to be very effective at reducing high blood pressure in several human trials. In a seven month test of elderly individuals with prehypertension taking one capsule a week of this composition reduced the normal systolic blood pressure in excess of 20%—22 mm Hg.

SENIOR CITIZEN BLOOD PRESSURE (BP) TABLE

| | Blood | |
|---|---|---|
| Date | Pressure | Comment |
| Dec. 16, 2007 | 128/79 | Ten Year Normal BP for the test subjects |
| | | (Prehypertension) |
| Feb. 27, 2008 | — | Started taking BP capsules |
| May 6, 2008 | 119/72 | After nine weeks systolic was down 9 mm Hg |
| Jun. 8, 2008 | 112/71 | After three months systolic was down 16 mm |
| | | Hg |
| Jul. 20, 2008 | 105/68 | After four months systolic was down 23 mm |
| | | Hg* |
| Aug. 9, 2008 | 103/63 | After five months systolic was down 25 mm |
| | | Hg |
| Aug. 17, 2008 | 108/72 | |
| Sep. 20, 2008 | 107/67 | |
| — | 106/68 | Average of last four BP measurements - |
| | | 20% reduction |

*After four months of taking this composition the reduction in normal BP leveled off to a constant value.

This composition is seen to have reduced the average normal systolic BP by 22 mm Hg and the diastolic BP by 11 mm Hg for prehypertension individuals. This BP reduction is equivalent to taking a combination of four of the medications, currently in use today, on a daily basis. While all of the current BP reduction medications require daily medication, some require several doses per day, this composition only requires taking a single small capsule once a week.

The mechanism of action is not well known but from test results it appears as though this composition improves the elasticity of the arteries allowing them to expand more easily. This was arrived at from the fact that the effects of strenuous exercise did not change before versus after taking this composition, and the fact that the level of cholesterol did not change before versus after taking this composition. These two facts rule out the possibilities that this composition made the heart weaker, or the composition cleaned out the arteries making more area for the blood to flow more easily. This conclusion is also supported by a second test discussed below.

In another test of middle aged individuals, with no hypertension history, taking one capsule a week, of the same composition as the prehypertension group, reduced the normal systolic blood pressure from an average norm of 120/62 to 102/64 over a six month period of taking the composition. In a test of middle aged individuals with no hypertension history taking one capsule a week of this composition reduced the normal systolic blood pressure in excess of 17%—18 mm Hg.

MIDDLE-AGED BLOOD PRESSURE (BP) TABLE

| | Blood | |
|---|---|---|
| Date | Pressure | Comment |
| Jun. 25, 2007 | 120/62 | Normal BP for the test subjects |
| Feb. 27, 2008 | — | Started taking BP composition capsules |
| Jul. 1, 2008 | 110/64 | After four months systolic was down 10 mm |
| | | Hg |
| Sep. 20, 2008 | 102/64 | After six months systolic was down 18 mm Hg |

This finding is extremely significant as it shows the amount of BP reduction is a function of the normal BP level to begin with and is not a fixed value as is found in current BP medications on the market. This finding agrees well with the mechanism of action that this composition is thought to improve the elasticity of the arteries.

The composition is thought to rejuvenate the artery wall cells and allow them to expand more easily than was initially possible causing the hypertension condition. It is thought, due to the four month time it takes to achieve its full effect, that the old layer of arterial surface cells needs to be replaced by new healthy ones before elasticity is fully restored.

In older adults the artery walls of high blood pressure patients are more rigid than in younger adults. While hardening of the arteries is less pronounced in younger adults, than in older adults, the reduction in BP for the younger group would be expected to be less than that seen with the older group, and this is just what was seen.

This finding indicates the reduction in normal BP would be significantly greater than 22 mm Hg, seen for individuals with prehypertension, for individuals with advanced hypertension, BP>140 mm Hg. As the reduction in BP is less in younger individuals with no hypertension history this composition would be safe for anyone to take no matter the their degree of hypertension including individuals with no-hypertension history.

From the test data an empirical formula was arrived at that predicts the six month systolic pressure level for various initial systolic blood pressures between 95 and 150 mm Hg.

BP=P*[1−(P−91)/220] mm Hg

Where, P=initial systolic blood pressure in mm Hg, and BP=six month systolic blood pressure (±5%). The following table summarizes the prediction for various initial BP.

SIX MONTH PREDICTED SYSTOLIC PRESSURE

| Initial Normal | Predicted Normal | Systolic Pressure | Tolerance |
|---|---|---|---|
| Systolic Pressure | Systolic Pressure | Reduction (mm Hg) | (± mm Hg) |
| 100 | 96 | 4 | 5 |
| 110 | 100 | 10 | 5 |
| 120 | 104 | 16 | 5 |
| 130 | 107 | 23 | 5 |
| 140 | 109 | 31 | 5 |
| 150 | 110 | 40 | 5** |

**Systolic blood pressures above 140 mm Hg requires taking two capsules a week.

It can be seen that this composition has the ability to replace all of the current medication available today with a single capsule a week. As this composition is composed of natural pure sugars it has no side effects and can be taken by anyone. The current alternative requires patients to take combinations of medication that each only reduces the BP by a very small amount.

Blood pressure reduction medications currently on the market are all very specific and all have adverse side effects associated with their use and none of the currently used medications can be taken by everyone without problems. This novel invention can be taken by everyone and has no adverse side effects or any adverse long term effects and its wide range of action will allow it to replace all of the medications on the market today.

### EXAMPLE 5

In a different embodiment of this invention another suitable composition for a product according to the present invention is shown in the following table:

| | Weight % | | |
|---|---|---|---|
| Carbohydrate | (range) | Weight % (tested) | Human (mg) |
| D-Mannose | 0.1 to 40 | 25.3 | 90 |
| D-Galactose | 0.1 to 30 | 11.3 | 40 |
| Sialic Acid | 0.1 to 70 | 15.5 | 55 |
| N-Acetyl-Galactosamine | 1.0 to 80 | 22.6 | 80 |
| Glucosamine HCL | 0.1 to 30 | 5.6 | 20 |
| L-Fucose | 0.5 to 75 | 19.7 | 70 |

In this combination the ingredients Glucosamine HCL and D-Mannose are optional and preferred. This composition was packaged in a size #1 capsule and taken once a week. It is good for both humans and animals.

This composition was found to be extremely effective at restoring system pH during a human tests conducted from January 2007 to July 2008. This carbohydrate combination needs to be taken twice a week, at bedtime, for system pH values above 6. Noticeable increases in pH were observed after two months and complete restoration to a pH value of 7, or above, was seen after five months of use. After taking the composition for two months the pH had improved from 6.5 to 6.7. This carbohydrate composition was derived from the results of the 2008 tests.

SYSTEM pH Restoration Table

| Date | System pH | Comment |
|---|---|---|
| Jan. 21, 2007 | 6.5 | |
| Feb. 24, 2008 | — | Started taking this composition |
| Apr. 19, 2008 | 6.7 | |
| Jul. 19, 2008 | 7.1 | |

The general treatment for low pH related conditions is the use of Calcium. Unfortunately, for lower pH levels Calcium absorption is greatly diminished and for pH levels below 5 this treatment is generally unavailable because of the toxic amount of calcium that need to be consumed on a daily basis.

One novelty of this invention is that it acts like a catalyst to change the pH level rather than the brute force calcium method and as such provides a new and safe method of raising system pH and thereby offering a new treatment for cancerous cells.

### EXAMPLE 6

In a different embodiment of this invention another suitable composition for a product according to the present invention is shown in the following table:

| | Weight % | | |
|---|---|---|---|
| Carbohydrate | (range) | Weight % (tested) | Human (mg) |
| L-Fucose | 0.1 to 50 | 25.0 | 90 |
| Glucosamine HCL | 0.1 to 25 | 22.2 | 80 |
| Sialic Acid | 0.1 to 30 | 13.9 | 50 |
| N-Acetyl-Galactosamine | 0.1 to 90 | 38.9 | 140 |

In this combination the ingredient Glucosamine HCL is optional and preferred. This composition is packaged in a size #1 capsule and is taken twice a week. It is good for both humans and animals in helping to restore normal glucose levels in the body's of borderline diabetics.

This composition was found to be extremely effective at reducing the high glucose levels of borderline diabetics during human tests conducted from March 2007 to June 2007. This carbohydrate combination needs to be taken twice a week, at bedtime. After taking the composition for five months the fasting glucose level dropped from a high of 117 down to 69. This drop is a 41% drop in fasting blood glucose levels. The normal range for glucose is from 65 to 100. Glucose levels from 100 to 125 is considered borderline diabetic. Currently there is no known medical treatment for a borderline diabetic.

Fasting GLUCOSE Levels

| Date | Glucose Level | Comment |
|---|---|---|
| Mar. 27, 2007 | 117 | |
| Apr. 13, 2007 | 94 | |
| Jun. 1, 2007 | 82 | |
| Jun. 28, 2007 | 86 | |

In a wider range test the average fasting blood glucose level was 90.5. After five months of using the composition the average fasting glucose level fell to 73.5. This represents a 23% drop in fasting glucose levels.

Fasting GLUCOSE Levels 2

| Date | Glucose Level | Comment |
|---|---|---|
| — | 90.5 | Averaged from Mar. 30, 2007 to |
| | | Feb. 29, 2008 |
| Feb. 27, 2008 | — | Started taking this composition |
| Jul. 17, 2008 | 69 | |
| Aug. 14, 2008 | 78 | |
| — | 73.5 | Average for last two glucose levels |

This composition was also tested on a second group of normal subjects who were not diabetics or borderline diabetics. Their average fasting glucose level was 82 for the group. After five months the average fasting glucose level was down to 71. The reduction in glucose levels for the normal group was 15%.

From the test results it can be seen that the composition does not affect individuals with normal glucose levels as much as it does individuals with high glucose levels. This important finding indicates that using this composition will not disproportionately reduce glucose levels to a dangerous level in normal individuals.

While other essential sugars/carbohydrates could have been added to this composition, such as Mannose, Galactose, and Xylose, to enhance the composition the ingredients shown in Example 7 are the least known to be therapeutically beneficial for the treatment of borderline diabetics as found from the 2007 and 2008 tests.

Another novelty of this invention is that it is able to restore normal glucose levels in borderline diabetics while being safe for non-diabetics to also take.

Another novelty of this invention is that it is able to restore normal blood pressure levels in prehypertension individuals while being safe for individuals to take who have no hypertension history.

Another novelty of the present invention is that it has no known side effects and it only needs to be administered once or twice a week. Up until now the few medications available needed to be taken on a daily basis and didn't produce lasting results or such pronounced results as this invention produces.

It can be seen that this invention has a large number of possible beneficial compositions utilizing just one, or any combination, of the seven essential sugars specialized for a specific target. Just because a combination is not specifically set out herein should not limit the scope of this invention. It has been sufficiently shown that there are numerous compositions available with useful purposes by the detailed examples set out herein.

Additionally, the weighting of a composition if varied by ingredient will specify the composition for a new target use even though the ingredients for two different target uses are the very same, refer to Examples 4 and 5. By changing the amounts of an ingredient its effects on cell absorption will change and by increasing or reducing an ingredient within a cell it will turn on or off different genes which will alter the body's response; The Geno Type Diet by Dr. Peter J. D'Adamo, Broadway Books, 2007, ISBN 978-0-7679-2524-2.

Marker Cancer Test.

What would be extremely useful, but has alluded everyone so far, is a way to quickly test for the conditions within the body that support cancer cell growth and thereby allow for early treatment of disease. Current technologies have devised ways to test for the presence of specific cancers, after they have formed and are growing, using many exotic and complicated techniques. What is needed is a simpler way to test for the body's conditions that can be used as an early warning marker long before cancer starts. Prior art has stopped short of such a technique but did indicate that saliva pH may hold the key.

The next logical jump would have been to do correlation studies to determine if there is any direct relation to saliva pH and cancer. By studying the related research literature it was found that cancer forming conditions and saliva pH were related.

This relation can be easily explained by the way the body protects itself. Consider an individual stranded in a very cold environment without adequate clothing. In defense the body cuts the blood circulation to its extremities first. This is done in order to keep the body's core temperature, and the brain's temperature, at near normal levels. As the body gets colder it further cuts off circulation to its extremities, and so on.

In the case of disease its not all that different just the systems to be shut down differ. The body's fluid systems (blood, saliva, lymphatic, spinal, etc.) are all buffered to keep their associated pH within a narrow region. As the body starts running out of the chemicals and nutrients required to maintain the pH of all of these systems it starts cutting the amount of regulation to the extremities first. The lowest priority system/extremity would be the body's saliva.

As the body runs out of the necessary building blocks to keep its system pH at a normal level disease sets in. As there is not enough chemicals and nutrients to keep all of the systems fully buffered the pH of the saliva is the first to drop, see Barefoot page 136.

However, just measuring the pH of a person's saliva will give a wide variance of readings. What else is needed is a process that will produce consistent pH measurement results for any given subject. The readings need to be reproducible to within 0.1 pH. There are electronic pH meters that have this accuracy and could be used but again there are problems.

First the digital unit will need to read to 0.01 pH so the random inaccuracies of the unit will be far below the 0.1 pH resolution needed. From research it was found that the more expensive pH units did not work as well as a lot of the less expensive units. This stems from the fact that most of the cheaper units required less saliva to give a reading while the more expensive units required more saliva. The cost trade off here is that the less expensive units require calibration before each use while the more expensive units don't.

Another way the saliva can be measured is by using pH test paper. It is made in 0.2 pH increments. If the color differences, produced by this paper, are made by a trained eye its possible to get 0.1 pH accuracies. The main problem with using pH test papers is their calibration to the color chart may be off by 1.5 pH. To get around this error the test paper is first calibrated to the color chart by dipping it in a 7.0 buffer solution and recording the reading difference (between what it indicates the pH is and 7.0 which is the true pH).

Now that a method of measuring the pH has been disclosed a method of when to take it is as important. After a lot of trials over the last several years the following is a method that produces consistent saliva pH results.

On first getting up in the morning, before washing the mouth out or having breakfast, ease a saliva sample from the mouth into a plastic tea spoon. If the saliva is spit into the spoon there will be air bubbles that will distort the reading and make it useless. This next part is important—do not start thinking about getting something to eat or smell any food being cooked before taking the sample. The thought that food is going to be eaten soon will change the pH of the saliva which will give a false indicating.

Using pH test paper will contaminate the saliva sample so if this method is used to measure the pH the spoon will need to be emptied out and cleaned after each test. If an electronic pH meter is used to measure the pH then the sample can be reused.

Once the pH of the saliva has been measured refer to the following table to determine the body's condition.

Marker Test Table

| Level | Saliva pH | Condition |
|---|---|---|
| 1 | Above 6.5 | Normal |
| 2 | 6.0-6.5 | This is an indicator that the body doesn't have all |
| | | it needs |
| 3 | 5.5-6.0 | This is the level where tumor cell growth can start |
| 4 | 5.0-5.5 | Cancer, tumor and neoplasia growth conditions are |
| | | very good |
| 5 | 4.5-5.0 | Detection stage where cancers like leukemia can be |
| | | detected |
| 6 | Below 4.5 | Cancer is in an advanced state in the body and growing |

Treatment for all of these levels/conditions is to simply raise the body pH back to normal. The current treatment for low body pH is taking Calcium and vitamin D supplements.
At Level 1 (pH around 6.5) one to two gram(s) a day of Calcium is required.
At Level 2 (pH around 6.0) Calcium treatment is losing its effectiveness.
Below pH 6.0 Calcium treatment becomes totally ineffective.
Level 4 (pH below 5.5) depending on type of cancer or tumor it may be detectable by other means by now.
Level 5 is where a number of the cancers have been detected and treatments like chemo is started.
By Level 6 the disease has progressed so far that death is the general prognosis.

As can be seen this new marker test will start to indicate problems within the body long before the actual disease is detectable by other means. This advanced notice also makes very early treatment possible. Currently, treatment doesn't start until the disease has progressed so far its life threatening. This novel marker test will eliminate delayed treatment and disease progression and allow time to replenish the chemicals and nutrients required by the body for proper health long before the conditions become life threatening.

In summary, this invention pertains to the field of dietary supplements and nutritional support for promotion and maintenance of optimal good health and a way to determine the body's condition and maintenance of it. More specifically, the invention relates to compositions of seven essential sugars/carbohydrates/glycoproteins as dietary supplements that are essential for a mammal's optimal health and functionality and a way to quantify and classify the body's condition and need for nutrients and or treatments.

This invention will correct the problem caused by modern diets consisting of highly refined foods, from which many essential ingredients have been eliminated during processing, specifically the seven essential sugars needed for a properly functioning

mammal. It will also cure the problem inherent in most of the glyconutrients available today that contain only trace amounts of these essential sugars while containing large amounts of inactive ingredients that can and do cause numerous allergic reactions with no benefit realized. It also sets out a novel approach to determining the body's state of health.

The above is a detailed description of particular embodiments of the invention. Those of skill in the art should, in light of the present disclosure, appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

Since other modifications and changes varied to fit particular operating requirements and environments will be apparent to those skilled in the art, the invention is not considered limited to the examples chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A composition of at least three or more carbohydrate(s) selected from the following group: galactose, mannose, n-acetylneuraminic acid, fucose, n-acetylgalactosamine, n-acetylglucosamine, xylose, glucosamine HCL, glucosamine sulfate, wherein said composition is used as a beneficial treatment to restore system pH and for pH related illnesses.

2. The compositions of carbohydrates in accordance with claim 1, wherein said composition further comprises a flowing agent or a lubricant or a filler ingredient.

3. The compositions of carbohydrates in accordance with claim 1, further comprising one or more non-toxic vitamins and or minerals.

4. The compositions of carbohydrates in accordance with claim 1, further comprising one or more non-toxic herbal, fungal, plant and or animal derived agents.

5. The compositions of carbohydrates in accordance with claim 1, used as a dietary supplement.

6. The compositions of carbohydrates in accordance with claim 1, administered to humans or animals using any of the following methods: capsule, caplet, tablet, liquid, suppository.

7. A composition of at least two or more carbohydrate(s) selected from the following group: galactose, mannose, n-acetylneuraminic acid, fucose, n-acetylgalactosamine, xylose, glucosamine HCL, glucosamine sulfate, wherein said composition is used as a beneficial treatment to restore normal glucose levels in borderline diabetics.

8. The compositions of carbohydrates in accordance with claim 7, wherein said composition further comprises a flowing agent or a lubricant or a filler ingredient.

9. The compositions of carbohydrates in accordance with claim 7, further comprising one or more non-toxic vitamins and or minerals.

10. The compositions of carbohydrates in accordance with claim 7, further comprising one or more non-toxic herbal, fungal, plant and or animal derived agents.

11. The compositions of carbohydrates in accordance with claim 7, used as a dietary supplement.

12. The compositions of carbohydrates in accordance with claim 7, administered to humans or animals using any of the following methods: capsule, caplet, tablet, liquid, suppository.

13. A method of detecting cancer in a subject using a marker test, comprising analysis of a subject's saliva pH, wherein the measurement of the saliva's pH is indicative of the level of disease in the subject.

14. The method of claim 13, wherein a very low saliva pH is associated with cancer of the lung, head and neck, colon, leukemia, oral, breast, kidney, prostate, bladder or pancreatic.

15. The method of claim 13, wherein a low saliva pH is associated with a tumor or a neoplasia.

16. A composition of at least two or more carbohydrate(s) selected from the following group: galactose, mannose, n-acetylneuraminic acid, fucose, n-acetylgalactosamine, n-acetylglucosamine, xylose, glucosamine HCL, glucosamine sulfate, wherein said composition is used as a beneficial treatment to reduce high blood pressure.

17. The method of claim 16, wherein said composition further comprises one or more non-toxic vitamins and or minerals.

18. The compositions of carbohydrates in accordance with claim 16, wherein said composition further comprises a flowing agent or a lubricant or a filler ingredient.

19. The compositions of carbohydrates in accordance with claim 16, further comprising one or more non-toxic herbal, fugal, plant and or animal derived agents.

20. The compositions of carbohydrates in accordance with claim 16, used as a dietary supplement.

21. The compositions of carbohydrates in accordance with claim 16, administered to humans or animals using any of the following methods: capsule, caplet, tablet, liquid, suppository.
